(19) United States
(12) Patent Application Publication
(10) Pub. No.: US 2015/0305367 A1
(43) Pub. Date: Oct. 29, 2015

(54) FROZEN CONFECTION

(71) Applicant: CONOPCO, INC., D/B/A UNILEVER, Englewood Cliffs, NJ (US)

(72) Inventors: Benjamin John MADDISON, Bedfordshire (GB); Charlotte Mary WALDEN, Market Harborough (GB); Joy Elizabeth WILKINSON, Bedfordshire (GB)

(73) Assignee: Conopco, Inc., d/b/a UNILEVER, Englewood Cliffs, NJ (US)

(21) Appl. No.: 14/443,809

(22) PCT Filed: Nov. 22, 2013
(86) PCT No.: PCT/EP2013/074451

§ 371 Date: May 19, 2015

(30) Foreign Application Priority Data
Nov. 23, 2012 (EP) 12194054.8

(51) Int. Cl. A23G 9/36; A61K 31/137; A61K 31/11; A61K 31/216; A61K 31/357

(52) U.S. Cl. CPC A23G 9/36; A61K 31/216; A61K 31/357; A61K 31/11; A61K 31/137; A23V 2002/00

(57) ABSTRACT

A frozen confection comprising from 0.002 wt % to 1 wt % of one or more compounds selected from the group consisting of benzyl cinnamate, piperonal, 4-methoxybenzaldehyde, and 4-hydroxybenzaldehyde is provided. A product for use in improving mood is also provided.

### TECHNICAL FIELD OF INVENTION

The present invention relates to a frozen confection that is capable of enhancing mood. More particularly, it relates to a frozen confection comprising novel inhibitors of the enzyme monoamine oxidase B.

### BACKGROUND OF INVENTION

Phenylethylamine (PEA) is a naturally occurring organic compound that functions in the brain as a neuromodulator and neurotransmitter in the mammalian central nervous system. It is present endogenously in mammals and is also found in many other organisms and in foods such as chocolate. Phenylethylamine potentiates neuronal responses to noradrenalin and dopamine, causing a sympathomimetic reaction and has been shown to have beneficial mood effects (Paterson et al., Journal of Neurochemistry 55(6):1827-37, 1990). Phenylethylamine is found throughout the central nervous system and studies have demonstrated the presence of phenylethylamine in the brain, in particular with relatively high levels in the limbic system, the emotion centre of the brain.

Frozen confections such as ice cream have also been shown to have an effect on the brain, in particular the orbitofrontal cortex, a part of the brain that is known to activate when people enjoy themselves (see for example “How ice cream tickles your brain”, The Guardian, Apr. 29 2005). The combination of ice cream with the psycho-pharmacological effect of phenylethylamine is therefore an especially suitable means for providing a mood enhancing food product.

However, orally ingested phenylethylamine is usually inactive because of extensive first-pass metabolism by the enzyme monoamine oxidase B (MAO-B) which breaks phenylethylamine down into phenylacetic acid. This prevents significant concentrations from reaching the brain.

There is therefore significant interest in molecules that are capable of inhibiting the breakdown of phenylethylamine by monoamine oxidase B. For example, U.S. Pat. No. 5,529,988 relates to substituted silyl alkylene amines and their pharmacological use as MAO-B inhibitors. EP0582825 relates to certain pyridine-2-carboxamides that are reversible and highly active MAO-B inhibitors. WO9215551 relates to certain propargylamines are useful as selective monoamine oxidase B inhibitors and that have neuroprotective properties in human and veterinary medicine.

Although these compounds are alleged to be capable of inhibiting MAO-B, they are not naturally occurring ingredients. It would not be acceptable to consumers to provide products that have such additives and they are therefore not suitable for inclusion in products such as frozen confections.

There therefore remains a need for specific, naturally occurring compounds that are suitable for incorporation with frozen confections such as ice cream and that are capable of inhibiting the breakdown of phenylethylamine by monoamine oxidase B.

### SUMMARY OF INVENTION

We have now found that a specific subset of compounds found in vanilla are capable of significantly inhibiting the activity of monoamine oxidase B. Since these compounds are sourced from this typical component of ice cream they represent a natural alternative to pharmaceutical-based MAO-B inhibitors.

Therefore, in a first aspect, the present invention provides a frozen confection comprising at least 0.002 wt % of one or more compounds selected from the group consisting of benzyl cinnamate, piperonal, 4-methoxybenzaldehyde, and 4-hydroxybenzaldehyde.

Preferably the frozen confection comprises at least 0.005 wt %, more preferably at least 0.010 wt %, more preferably still at least 0.015 wt %, yet more preferably at least 0.02 wt %, yet more preferably still at least 0.05 wt %, most preferably at least 0.1 wt % of one or more compounds selected from the group consisting of benzyl cinnamate, piperonal, 4-methoxybenzaldehyde, and 4-hydroxybenzaldehyde.

The frozen confection comprises at most 1 wt % of one or more compounds selected from the group consisting of benzyl cinnamate, piperonal, 4-methoxybenzaldehyde, and 4-hydroxybenzaldehyde.

Preferably the frozen confection comprises at most 0.5 wt %, more preferably at most 0.4 wt %, more preferably still at most 0.3 wt %, yet more preferably at most 0.2 wt % of one or more compounds selected from the group consisting of benzyl cinnamate, piperonal, 4-methoxybenzaldehyde, and 4-hydroxybenzaldehyde.

Preferably the compound selected is benzyl cinnamate.

Phenylethylamine can be present endogenously and therefore need not be present in the frozen confection. However, the product can also provide additional phenylethylamine. Therefore the frozen confection preferably comprises at least 0.00001 wt % phenylethylamine, more preferably at least 0.0001 wt %, more preferably still at least 0.001 wt %, yet more preferably still at least 0.01 wt %.

Preferably the frozen confection comprises at most 0.5 wt % phenylethylamine, more preferably at most 0.1 wt %, more preferably still at most 0.05 wt %.

In a second aspect, the invention provides the product of the first aspect for use in improving mood.

### DETAILED DESCRIPTION OF INVENTION

Phenylethylamine is a highly lipid-soluble, brain-permeable amine which easily crosses the blood-brain barrier and is in dynamic equilibrium between central and peripheral compartments. It is found in trace amounts throughout the brain at concentrations of roughly 2 nM. Exogenous phenylethylamine is found in various foods, including chocolate, mature cheeses and sausages. It can also be synthesised endogenously from L-phenylalanine by the aromatic L-amino acid decarboxylase. It can therefore be formed in all tissues capable of taking up L-phenylalanine and containing aromatic L-amino acid decarboxylase.

Physiologically, phenylethylamine potentiates neuronal responses to noradrenalin and dopamine, causing a sympathomimetic reaction, i.e. an amphetamine-like response. It is also believed to function through trace amine-associated receptor 1 which is implicated in regulation of dopamine signalling. Phenylethylamine has been shown to have mood effects and its systemic administration in animals produces amphetamine-like effects (Mantegazza & Riva, Journal of Pharmacology 15: 472-478, 1963).

Phenylethylamine is rapidly metabolized, predominantly via monoamine oxidase B (MAO-B) and to a lesser extent by monoamine oxidase A (MAO-A). Consequently, phenylethylamine has a fast turnover rate as demonstrated by a very brief endogenous pool half-life of approximately 30 s as measured in vivo in rat brain. Both endogenous and exogenous phenylethylamine in humans is primarily metabolised to phenylacetic acid.

The enzyme monoamine oxidase (Enzyme Classification 1.4.3.4.) is located on the cytosolic face of outer mitochondrial membranes and catalyzes the oxidative deamination of amines from both endogenous and exogenous sources. In addition to phenylethylamine, its substrates include the neurotransmitters serotonin, norepinephrine, dopamine, and tyramine. Monoamine oxidase has two isoenzymes, with different sensitivities to known inhibitors and with different substrate specificities. The MAO-B isoform is the major degradative isoform for phenylethylamine. It is found in brain and in the periphery (heart, liver, kidney, intestine, blood platelets and lymphocytes).

It is therefore readily apparent that the beneficial mood effects associated with both endogenous and exogenous phenylethylamine are adversely affected by the activity of MAO-B. In fact, pharmacological mood effects are seen only at very high doses of phenylethylamine or following pre-treatment with MAO-B inhibitors. Studies have shown that when depressed subjects were administered with phenylethylamine (10-60 mg/day) or its precursor L-phenylalanine (250 mg/day) in conjunction with the selective MAO-B inhibitor deprenyl (5-10 mg/day), a significant number of patients reported relief of depression and improvement in mood.

Due to the role played by MAO-B in the degradation of neurotransmitters, pharmaceutical MAO-B inhibitors have long been investigated for potential therapeutic uses. MAO-B inhibitors reduce oxidative metabolism of dopamine in the brain and are used as neuroprotective agents in treatment of Parkinson's disease. In the study of the pharmacokinetics and pharmacodynamics of MAO-B inhibitors, it has been shown in animal and human studies that MAO-B inhibition increases plasma, urine and brain levels of endogenous phenylethylamine. Studies in animals have shown that peripheral administration of from 0.3 mg/kg to 1 mg/kg of the pharmaceutical MAO-B inhibitors Selegiline or Mofegiline increase brain levels of phenylethylamine 10 to 100 fold, depending on brain region and dose.

Despite the interest in pharmaceutical MAO-B inhibitors, there remains a need for natural alternatives that can be delivered to consumers through everyday products. Frozen confections such as ice cream are one such everyday product and have also been shown to have an effect on the brain, in particular the orbitofrontal cortex which is a part of the brain known to activate when people enjoy themselves. The combination of ice cream with the psycho-pharmacological effect of phenylethylamine would therefore be an especially suitable product format. However, any exogenous phenylethylamine in such a consumer product would still be subject to degradation by MOA-B and any addition of existing pharmaceutical MAO-B inhibitors to overcome this would not be acceptable. There is therefore a need for natural, non-pharmaceutical compounds suitable for incorporation with frozen confections that are also capable of inhibiting the breakdown of phenylethylamine by monoamine oxidase B.

Following extensive research into the components of vanilla, certain actives have been identified which demonstrate high levels of inhibitory activity against monoamine oxidase B. These particular actives are:

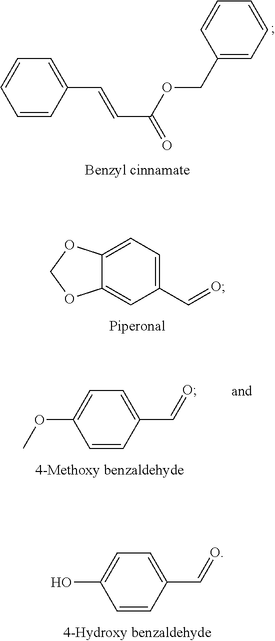

Benzyl cinnamate is also known as: Benzyl 3-phenyl propenoate; Phenyl Methyl 3-phenyl-2-propenoate; 3-Phenyl-2-Propenoic Acid Phenylmethyl Ester; Benzyl γ-phenylacrylate; Benzyl alcohol cinnamic ester; Benzyl alcohol, cinnamate; Cinnamein; Cinnamic acid, benzyl ester; Benzylester kyseliny skoricove; trans-Cinnamic acid benzyl ester; FEMA 2142; Phenylmethyl cinnamate; and Benzyl (E)-3-phenylprop-2-enoate.

Piperonal is also known as: Heliotropin; Heliotropine; Piperonyl aldehyde; Protocatechuic aldehyde methylene ether; and 3,4-methylenedioxybenzaldehyde.

4-Methoxybenzaldehyde is also known as: Anisal; Methyl-p-oxybenzaldehyde; Obepin; p-formylanisole; p-methoxybezaldehyde; para anisaldehyde; para anisic aldehyde; fema 2670; aubepine; anisaldehyde; and 4-anisaldehyde.

4-Hydroxybenzaldehyde is also known as: 4-formylphenol; p-formylphenol; p-hydroxybenzaldehyde; 4-hydroxybenzenecarbonal; p-oxybenzaldehyde; USAF m-6; 4-(hydroxyphenyl)methanal.

As mentioned, the compounds of the present invention are components of vanilla. Vanilla is a well known component of frozen confections and has been widely investigated. EP2206438 discloses a frozen confection or a beverage product is provided which contains at least 400 mg of theobromine and at least 40 mg of caffeine per 100 grams of the product. The frozen confection may contain vanilla flavouring. U.S. Pat. No. 4,099,531 discloses 2-Phenyl-3-(fur-2-yl)-prop-2-en-1-al as being useful in flavoring both tobacco and tobacco substitute materials as well as foodstuffs and beverages in general. Example 2 of this document discloses 25 parts heliotropin (piperonal) in 1000 parts of a base compound. U.S. Pat. No. 5,082,682 discloses a nonfat dairy dessert composition which may be aerated and frozen, containing milk solids nonfat, water, sweetener, a starch hydrolysate, egg albumen stabilizer and emulsifier. It also discloses the use of vanilla flavouring. WO2006/087370 discloses the use of aroma glycosides as flavor or fragrance ingredients. US2004/151816 discloses a no sugar-added soft serve ice cream composition comprising erythritol, full fat milk, cream, skim milk, stabilizers, egg yolk, vanilla extract, sucralose, and a component selected from maltodextrin, alkalized cocoa powder, chocolate liquor, or a blend of cocoa powder and chocolate liquor. The use of a vanilla extract is disclosed. U.S. Pat. No. 2,679,458 discloses a dry powder-like base material for making a frozen confection. The use of a vanilla flavour is mentioned. WO2012/107206 relates to the use of microcarpalide or a derivative or a stereoisomer or a salt or a hydrate thereof as a sweetener and/or a sweetness enhancer. Piperonal is mentioned as an aldehyde flavouring. U.S. Pat. No. 4,631,196 discloses a low cholesterol, low calorie, no fat dairy product and mentions the use of natural vanilla for flavouring. US2009/124701 discloses an individual alkamide and/or a mixture having two or more different alkamides for changing, masking or reducing the unpleasant flavor impression of an unpleasant-tasting substance or mixture of substances. JP10042826 is directed towards obtaining a Wasabia japonica flavor food capable of sufficiently giving the characteristic sweet smell of newly ground Wasabia japonica and holding good preservability. CN101991082 relates to a cream flavor food additive. 4-Methoxybenzaldehyde is mentioned. EP1806058 discloses the use of Decalipis hamiltonii or 2-hydroxy-4-methoxy benzaldehyde for use in combination with vanilla for flavoring foodstuffs. EP1066824 relates to a stimulative perfume composition which uses an anisaldehyde as the stimulative agent.

The foregoing documents mention the use of vanilla in frozen confections and in certain cases the compounds of the present invention are specifically named. However the documents do not provide sufficient information to be able to determine the levels of these actives in any frozen confections. In addition, these documents do not disclose that the levels of the compounds of the present invention should be elevated above normal levels. Moreover, none of these documents have discovered that the compounds of the present invention demonstrate high levels of inhibitory activity against monoamine oxidase B.

WO96/26720 discloses a pharmaceutical composition for oral administration comprising a carrier and, as an active ingredient, a monoamine oxidase B inhibitor, characterised in that the composition is formulated to promote pre-gastric absorption of said monoamine oxidase B inhibitor. WO92/15551 discloses aliphatic propargylamines as specific monoamine oxidase B inhibitors. EP0582825 discloses a process for preparing pyridine 2-carboxamides of a particular formula in which the R group is a known compound which is a monoamine oxidase B inhibitor. Although these documents discuss the use of certain compounds as monoamine oxidase B inhibitors, these too have not discovered that the compounds of the present invention demonstrate high levels of inhibitory activity against monoamine oxidase B.

The invention therefore provides a frozen confection comprising novel, elevated levels of the compounds of the present invention. In particular, the invention provides a frozen confection comprising from at least 0.002 wt % of one or more compounds selected from the group consisting of benzyl cinnamate, piperonal, 4-methoxybenzaldehyde, and 4-hydroxybenzaldehyde.

Preferably the frozen confection comprises at least 0.005 wr/o, more preferably at least 0.010 wt %, more preferably still at least 0.015 wr/o, yet more preferably at least 0.020 wt %, most preferably at least 0.050 wt % of one or more compounds selected from the group consisting of benzyl cinnamate, piperonal, 4-methoxybenzaldehyde, and 4-hydroxybenzaldehyde.

The frozen confection comprises at most 1 wt % of one or more compounds selected from the group consisting of benzyl cinnamate, piperonal, 4-methoxybenzaldehyde, and 4-hydroxybenzaldehyde. Preferably the frozen confection comprises at most 0.5 wt %, more preferably at most 0.4 wt %, more preferably still at most 0.3 wt %, yet more preferably at most 0.2 wt % of one or more compounds selected from the group consisting of benzyl cinnamate, piperonal, 4-methoxybenzaldehyde, and 4-hydroxybenzaldehyde.

Phenylethylamine can be present endogenously in the consumer of the product and therefore need not be present in the frozen confection. However, the frozen confection can also provide additional phenylethylamine and a preferred embodiment also comprises from 0.00001 wt % to 0.5 wt % phenylethylamine.

Frozen confections are sweet-tasting fabricated foodstuffs intended for consumption in the frozen state (i.e. under conditions wherein the temperature of the foodstuff is less than 0° C., and preferably under conditions wherein the foodstuff comprises a significant amount of ice). Frozen confections include water ices and fruit ices, which comprise water and one or more of sugars, stabilisers, colours and flavours, but little or no fat or protein (e.g. less than 5 wt % of each, preferably less than 2 wt %). Frozen confections also include ice creams, frozen yoghurts, sorbets and the like.

The frozen confection may be aerated or unaerated. The extent of the aeration can be measured in terms of the volume of the aerated product. The extent of aeration is typically defined in terms of “overrun”. In the context of the present invention, % overrun is defined in volume terms as:

$$Overrun ( \% ) = \frac{( volume of final aerated product - volume of unaerated mix )}{volume of unaerated mix} \times 100$$

If the frozen confection is aerated, the overrun is preferably at least 20%, more preferably at least 50%. It is preferable that the overrun does not exceed 200%, more preferably the overrun is less than 130%. Overrun is typically produced by intentionally incorporating gas into the product, such as by mechanical agitation. The gas can be any food-grade gas such as air, nitrogen or carbon dioxide.

The present invention will now be further described with reference to the following non-limiting examples.

### Examples

Eighteen test compounds as shown in table 1 were analysed for their ability to inhibit MAO-B. All test compounds, as well the positive control (deprenyl), were purchased from Sigma-Aldrich. These test compounds are all present in natural vanilla. Natural vanilla contains over 400 different compounds but the 18 compounds represented in table 1 are those that have a role in providing vanilla flavour.

TABLE 1
Test compounds
Test Compounds

4-Hydroxybenzyl alcohol
Vanillyl Alcohol
3,4-Dihydroxybenzaldehyde
4-Hydroxybenzoic acid
Vanillic acid
4-Hydroxybenzaldehyde
Vanillin
Coumaric acid
Ferulic acid
3-Hydroxy-2-butanone
4-Methoxybenzaldehyde
4-Hydroxy-2,5-dimethyl-3(2H)-furanone
2-Methoxyphenol
Benzyl cinnamate
Eugenol
Veratraldehyde
Ethyl vanillin
Piperonal

MAO inhibition potencies of the test compounds were examined using the chemiluminescent assay MAO-Glo™ (Promega) as described in the Valley, M., et al. article “A bioluminescent assay for monoamine oxidase activity”, Anal. Biochem. 359, 238-46 (2006). Microsomes derived from baculovirus-infected insect cells expressing recombinant human MAO-B (Sigma-Aldrich) were used as enzyme sources. Assays were carried out in 96-well black, solid plates according to manufacturers' instructions in which: MAO-B substrate was incubated for 90 minutes with the test compound and MAO-B enzyme (5 μg per reaction) in the total volume of 50 μl. Luminogenic MAO substrate, (45)-4,5-dihydro-2-(6-hydroxybenzothiazolyl)-4-thiazole-carboxylic acid, was used at concentrations of 40 μM and 4 μM, corresponding to Km values determined by the manufacturer for MAO-B. Triplicate reactions were performed in reaction buffer provided with the kit, at room temperature. To initiate a luminescent signal, 50 μl of luciferin detection reagent was added per reaction and incubated for a further 20 minutes to stabilize the signal. Luminescent light was recorded using the FLUOstar Omega plate reader (BMG Labtech) with a measuring time of 1 s for each well and gain set to 4095. Readings were displayed as relative light units (RLU). All test substances were dissolved in DMSO and diluted to the working concentration in reaction buffer, an equivalent concentration of DMSO was used as a negative control. 5 μM deprenyl (selective MAO-B inhibitor) was used as a positive control for inhibition. Concentrations of 1, 10 and 100 μM of the test compounds were chosen. For quantification of MAO inhibition and potency of test substances, results are expressed as the percentage MAO inhibition after the exposure to test compound, relative to the control MAO activity in the absence of inhibitor. To determine 1050 values (concentrations inhibiting 50% of maximum MAO-B activity), dose-response experiments were conducted at up to eight concentration steps (two-fold dilution ranging from 200-1.56 μM). Data was analysed using non-linear regression model by plotting logarithm of inhibitor concentration versus percentage MAO-B activity in GraphPad Prism software (version 5, GraphPad Software, US). Results are shown in table 2. The results are expressed as mean percentage MAO-B inhibition after exposure to respective concentration of test compound (μM)±standard deviation.

TABLE 2
Results of inhibition assays

| | Test | Average % | |
|---|---|---|---|
| | Concentration | MAO-B | MAO-B |
| Test compound | (μM) | inhibition | inhibition |
| 4-Hydroxybenzyl alcohol | 1 | 1 ± 7.9 | No |
| | 10 | 0 ± 7.8 | |
| | 100 | 6 ± 8.1 | |
| Vanillyl Alcohol | 1 | 2 ± 4 | No |
| | 10 | 9 ± 3 | |
| | 100 | 6 ± 5.1 | |
| 3,4-Dihydroxybenzaldehyde | 1 | 0 ± 1.3 | No |
| | 10 | 4 ± 5.1 | |
| | 100 | 9 ± 3.6 | |
| 4-Hydroxybenzoic acid | 1 | 13 ± 3.2 | No |
| | 10 | 9 ± 5.2 | |
| | 100 | 11 ± 5.7 | |
| Vanillic acid | 1 | 6 ± 4.2 | No |
| | 10 | 5 ± 7.3 | |
| | 100 | 11 ± 3.3 | |
| 4-Hydroxybenzaldehyde | 1 | 10 ± 3.2 | Yes |
| | 10 | 6 ± 4.7 | |
| | 100 | 27 ± 5.1 | |
| Vanillin | 1 | 7 ± 7.2 | No |
| | 10 | 8 ± 7.2 | |
| | 100 | 19 ± 5.6 | |
| Coumaric acid | 1 | 11 ± 4.2 | No |
| | 10 | 16 ± 2.8 | |
| | 100 | 17 ± 7.3 | |
| Ferulic acid | 1 | 4 ± 1.2 | No |
| | 10 | 9 ± 1.7 | |
| | 100 | 4 ± 7 | |
| 3-Hydroxy-2-butanone | 1 | 11 ± 4.2 | No |
| | 10 | 6 ± 5.2 | |
| | 100 | 7 ± 3.4 | |
| 4-Methoxybenzaldehyde | 1 | 0 ± 1.6 | Yes |
| | 10 | 11 ± 3.8 | |
| | 100 | 75 ± 1.9 | |
| 4-Hydroxy-2,5-dimethyl- | 1 | 0 ± 3.4 | No |
| 3(2H)-furanone | 10 | 9 ± 3.3 | |
| | 100 | 7 ± 11.7 | |
| 2-Methoxyphenol | 1 | 0 ± 2.8 | No |
| | 10 | 0 ± 0.5 | |
| | 100 | 0 ± 4.9 | |
| Benzyl cinnamate | 1 | 10 ± 6.4 | Yes |
| | 10 | 63 ± 3.1 | |
| | 100 | 100 ± 0 | |
| Eugenol | 1 | 0 ± 3.1 | No |
| | 10 | 4 ± 4.6 | |
| | 100 | 0 ± 4.2 | |
| Veratraldehyde | 1 | 1 ± 1.5 | No |
| | 10 | 2 ± 6.6 | |
| | 100 | 0 ± 2.5 | |
| Ethyl vanillin | 1 | 0 ± 4.4 | No |
| | 10 | 0 ± 4.6 | |
| | 100 | 4 ± 5.3 | |
| Piperonal | 1 | 10 ± 2.9 | Yes |
| | 10 | 53 ± 4 | |
| | 100 | 100 ± 0 | |

The foregoing results demonstrate that although the 18 compounds tested all have a role in providing vanilla flavour, benzyl cinnamate, piperonal, 4-methoxybenzaldehyde, and 4-hydroxybenzaldehyde are unique in their ability to inhibit MAO-B.

In a further experiment, the levels of benzyl cinnamate, piperonal, 4-methoxybenzaldehyde, and 4-hydroxybenzaldehyde in 6 typical ice cream formulations were measured. The 6 ice creams were: Wall's Cream of Cornish; Cremissimo Vanille; Carte d′Or Vanilla; Tesco Vanilletta; Nestlé la Laitière; and Sainsbury's Vanilla. A standard addition quantification method was used in which a known amount of target compound was added in a series of increasing steps to each ice cream sample. Samples were analyzed using standard GC-MS equipment with Poly-Acryllate (PA) Solid Phase Micro Extraction (SPME) as sample introduction technique. Samples were heated for 30 minutes at 60° C. while agitating in an automated system. Evaporating volatiles were trapped on a PA SPME fibre. After 30 minutes the fibre was thermally desorbed in a hot (250° C.) injector, allowing the volatiles to enter a GC-MS system. The volatiles were separated on a GC equipped with a Carbowax column and the temperature program used was: 40° C. (5 min) (−5° C./min)→250° C. (5 min). Separated compounds were detected with a mass spectrometer, which was used both for compound identity conformation and also for quantification. Quantification was done by calculating the peak areas of selected ions per compound. Before a reliable quantification by standard addition could be carried out, a rough estimation of the amount of target compound in a subset of the ice cream samples was made by comparing the response of a known amount of compound in water to the response of the compound in a sample. Using the estimated values, a series of additions was created with 0; 1; 2; 4; 6; 8 and 10 times the estimated amount of compound in each ice cream sample. Analysis of the samples using the described method resulted in a response curve for each compound in each sample, allowing the amount of compound present to be calculated. This was repeated for all compounds and all ice cream samples. The results are provided in Table 3 in which errors are calculated from the standard deviation of replicates.

TABLE 3
Levels of 4-hydroxy benzaldehyde, 4-Methoxy-benzaldehyde,
Piperonal, and Benzyl cinnamate in standard ice creams

| | Wall's | | | | | |
|---|---|---|---|---|---|---|
| | Cream | | Carte | | Nestlé | |
| Conc'n | of | Cremissimo | d'Or | Tesco | la | Sainsbury's |
| (μg/g [ppm]) | Cornish | Vanille | Vanilla | Vanilletta | Laitière | Vanilla |
| 4-hydroxy | <0.05 | 1 ± 0.6 | <0.05 | 2 ± 0.8 | 3 ± 0.8 | 3 ± 0.8 |
| benzaldehyde | | | | | | |
| 4-Methoxy- | 0.3 ± 0.2 | <0.1 | <0.1 | <0.1 | 0.4 ± 0.3 | <0.1 |
| benzaldehyde | | | | | | |
| Piperonal | 0.13 ± 0.05 | <0.05 | 0.07 ± 0.05 | 9 ± 0.6 | 15 ± 0.7 | <0.05 |
| Benzyl | <2 | <2 | <2 | <2 | <2 | <2 |
| cinnamate | | | | | | |

As can be readily appreciated from the results of Table 3, there is wide variation in the amounts of the compounds of the present invention that can be found in standard ice cream. However, even the highest levels (for example Piperonal in Nestlé la Laitière) are far lower than the levels claimed in the present invention.

1. A frozen confection comprising from 0.002 wt % to 1 wt % of one or more compounds selected from the group consisting of benzyl cinnamate, piperonal, 4-methoxybenzaldehyde, and 4-hydroxybenzaldehyde.

2. A frozen confection according to claim 1 comprising from 0.005 wt % to 0.5 wt % of one or more compounds selected from the group consisting of benzyl cinnamate, piperonal, 4-methoxybenzaldehyde, and 4-hydroxybenzaldehyde.

3. A frozen confection according to claim 1 or claim 2 wherein the compound selected is benzyl cinnamate.

4. A frozen confection according to any of the preceding claims comprising from 0.00001 wt % to 0.5 wt % phenylethylamine.

5. A product according to any of claims 1 to 4 for use in improving mood.
